(19) United States
(12) Patent Application Publication
(10) Pub. No.: US 2004/0034225 A1
(43) Pub. Date: Feb. 19, 2004

(54) Aryl-substituted n, n-heterocyclic compounds, method for their preparationand their use in therapeutics and diagnostics

(76) Inventors: Jacqueline Marchand-Brynaert, Marcinelle (BE); Jean-Francois Cavalier, Marguerittes (FR); Jean-Francois Rees, Hevillers (BE); Catherine De Tollenaere, Louvain-la-Neuve (DE); Maggi Burton, Vedrin-Namur (BE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/276,398

(22) PCT Filed: May 16, 2001
(86) PCT No.: PCT/EP01/05588

(30) Foreign Application Priority Data
May 17, 2000 (EP) 00870107.0
Dec. 12, 2000 (EP) 00870293.8

(51) Int. Cl. C07D 241/02

(52) U.S. Cl. 544/336

(57) ABSTRACT

The present invention relates to an aryl substituted pyrazine compound of the general formula I, II, III or IV with the exception of a) 2-amino-3,5-bis(p-methoxyphenyl)-1,4-pyrazine (CD29), 2-amino-5-phenyl-1,4-pyrazine (CD12), 2-amino-5-(4-methoxyphenyl)-1,4-pyrazine (CD17) and 2-amino-5-(4-hydroxyphenyl)-1,4-pyrazine (CD22) and of b) their corresponding imidazolopyrazinone compounds. Another aspect of the invention relates to anti-oxidant compounds of formula V. Another aspect of the invention is a compound which upon oxidation results via a cascade in a second anti-oxidant compound and a third compound.

The invention relates in general to aryl-substituted pyrazine compounds and the corresponding imidazolopyrazinone compounds having an anti-oxidant activitiy.

Several publications demonstrated that imidazolopyrazinone compounds, which are analogues of the natural coelenterazine (CLZn) are endowed with to antioxidative properties (Rees, J.-F. et al., J. Exp. Biol., 1998, 201, 1211-1221). It has been shown to scavenge a wide range of reactive oxygen species (ROS) such as singlet oxygen, superoxide anion, peroxynitrite, hydroxyl, alkoxyl and peroxyl radicals and so prevent free-radical-induced lipid peroxidation in cellular and a cellular systems. In addition, the reaction of coelenterazine-like imidazolopyrazinones generates coelenteramine-like aminopyrazines (CLM) which also possess chain-breaking properties as shown hereunder:

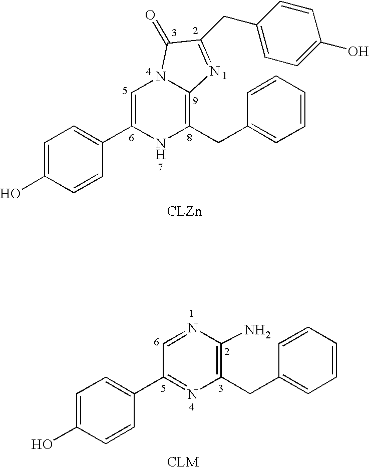

As an example the utilization of both aminopyrazine and imidazolopyrazinone compounds (related to the natural CLZn and CLM) as antioxidants are described in WO 96/28160 and WO 98/43641.

An object of the present invention is to provide novel aryl-substituted pyrazine compounds and the corresponding imidazolopyrazinone compounds having antioxidant properties. A further object is to provide in a simple way said novel compounds.

Another object is to provide antioxidative compounds having an easely tuneable lipophilicity.

Surprisingly four specific related structures of aryl-substituted pyrazine compounds as claimed in claim 1 were found having an unexpectedly high antioxidant activity.

The compounds of formula (I-II-III-IV) are deemed novel provided that 2-amino-3,5-bis(p-methoxyphenyl)-1,4-pyrazine (CD29), 2-amino-5-phenyl-1,4-pyrazine (CD12), 2-amino-5-(4-methxoyphenyl)-1,4-pyrazine (CD17) and 2-amino-5-(4-hydroxyphenyl)-1,4-pyrazine (CD22) and the corresponding imidazolopyrazinone compounds are not included and thus the present invention also relates to the compounds of formula (I-II-III-IV) as defined here-above provided that 2-amino-3,5-bis(p-methoxyphenyl)-1,4-pyrazine (CD29), 2-amino-5-phenyl-1,4-pyrazine (CD12), 2-amino-5-(4-methoxyphenyl)-1,4-pyrazine (CD17), 2-amino-5-(4-hydroxyphenyl)-1,4-pyrazine (CD22), 5-phenyl-2-methylamino-1,4-pyrazine and 2-amino-3,5-bis-phenyl-1,4-pyrazine and their corresponding imidazolopyrazinone compounds are not included. These known compounds are described in an electronic conference, available on the internet, “Synthesis of 3,5-disubstituted 2-aminopyrazines by palladium-mediated cross-couplings and its use for preparing chemi- and/or bioluminiescent compounds” by Hideshi Nakamura et al. and in the publications: H. Nakamura et al., Synlett, 1995, 1227-8 and K. Teranishi et al., Carbohydr. Res. 1998, 306,177-187.

The term prodrug as used throughout this text means the pharmacologically acceptable derivatives, e.g. esters and amides, such that the resulting biotransformation product of the derivative is the active drug as defined in the compounds of formula (I-II-III-IV). The reference by Goodman and Gilman (The Pharmacological Basis of Therapeutics, 8^{th} ed., McGraw-Hill, Int. Ed. 1992, “Biotransformation of Drugs”, p. 13-15) describing prodrugs generally, is hereby incorporated.

In another aspect the invention is also related to compounds of the general formula V:

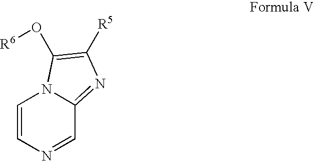

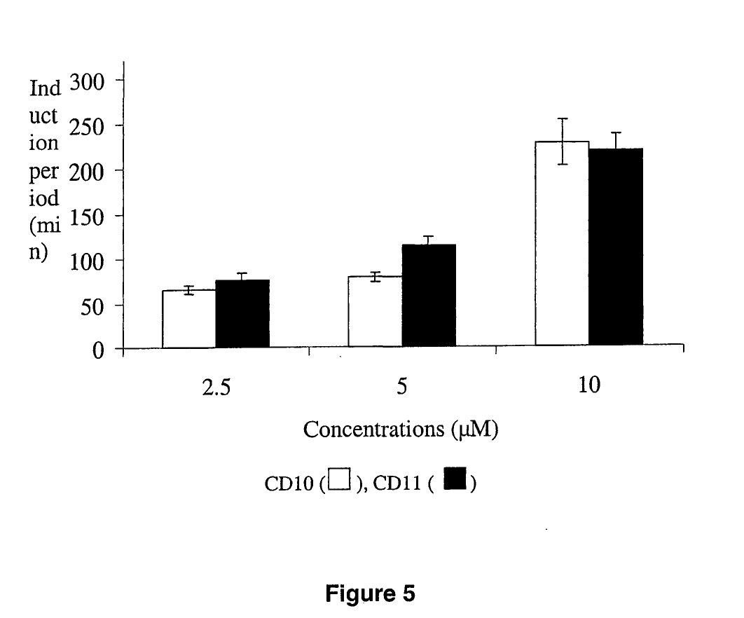

their pharmaceutical acceptable addition salts, a stereochemically or tautomerically isomeric form thereof, for use as a medicament, in particular having an anti-oxidant activity. It relates in particular to a family of imidazolopyrazinones corresponding to the general formula V, wherein R⁵ and R⁶ are defined as in formula II. Nine compounds are already known, but the anti-oxidative activities and potential medical applications of the family are novel (see FIG. 5 with CD10 and CD11). Hereunder these preferred anti-oxidant embodiments of the general formula V are illustrated wherein R⁶=H:

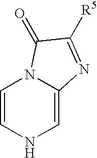

R⁵=Me (CD10) [RN=19943-97-6]

R⁵=Ph (CD11) [RN=27955-58-4]

R⁵=tBu [RN=152916-61-5]

R⁵=Et [RN=57683-97-3]

R⁵=PhpCl [RN=123488-69-7]

R⁵=PhpOMe [RN=123488-68-6]

R⁵=CH₂Ph [RN=144763-52-0]

R⁵=CH₂PhpOMe [RN=152719-89-6]

R⁵=CH₂PhpCF₃ [RN=152719-90-9]

As used herein C₁₋₆ alkyl as a group or part of a group defines straight or branched chain saturated hydrocarbon radicals having from 1 to 6 carbon atoms such as methyl, ethyl, propyl, 1-methylethyl, butyl, pentyl, hexyl, 2-methylpropyl, 2-methylbutyl and the like; C₁₂₋₁₈ alkyl as a group or part of a group defines straight or branched chain saturated hydrocarbon radicals having from 12 to 18 carbon atoms such as the groups defined for C₁₋₆ alkyl and C₁₂₋₁₈ alkenyl used as the groups defined for C₁₂₋₁₈ alkyl, but having one or more sites of unsaturation.

When any variable (e.g. aryl, R¹, R², R³ etc.) occurs more than one time in any constituent, each definition is independent. The aforementioned numbers, besides the ring structures, are merely illustrative for a better chemical comprehension, and not related to the subscript above the R substituents.

It will be appreciated that some of the compounds of formula (I-II-III-IV) and their prodrugs, addition salts, quaternary amines and stereochemically isomeric forms may contain one or more centers of chirality and exist as stereochemically isomeric forms.

The term “stereochemically isomeric forms” as used hereinbefore defines all the possible stereoisomeric forms which the compounds of formula (I-II-III-IV), and their prodrugs, addition salts, physiologically functional derivatives may possess. Unless otherwise mentioned or indicated, the chemical designation of compounds denotes the mixture of all possible stereochemically isomeric forms, said mixtures containing all diastereomers and enantiomers of the basic molecular structure as well as each of the individual isomeric forms of formula (I-II-III-IV) and their prodrugs, salts, solvates are obviously intended to be embraced within the scope of this invention.

For therapeutic use, salts of the compounds of formula (I-II-III-IV) are those wherein the counterion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not are included within the ambit of the present invention.

The pharmaceutically acceptable acid and base addition salts as mentioned hereinabove are meant to comprise the therapeutically active non-toxic acid and base addition salt forms which the compounds of formula (I-II-III-IV) are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, ptoluenesulfonic, cyclamic, salicylic, p-aminosalicylic, pamoic and the like acids.

Conversely said salt forms can be converted by treatment with an appropriate base into the free base form.

The compounds of formula (I-II-III-IV) containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term addition salt as used hereinabove also comprises the solvates which the compounds of formula (I-II-III-IV) as well as the salts thereof, are able to form. Such solvates are for example hydrates, alcoholates and the like.

Some of the compounds of formula (II and IV) may also exist in their tautomeric form. Such forms although not explicitly indicated are intended to be included within the scope of the present invention.

The definition of R³ and R⁵ are defined in the present invention in a functional way in order to comprise all the possible radicals. R³ is H or an alkylating reagent useful in the synthetic scheme B; preferentially benzyl and substituted benzyl, C₁₋₆ alkyl and branched and optionally substituted C₁₋₆ alkyl with carboxyl functions and derived functions, C₁₂₋₁₈ alkyl, C₁₂₋₁₈ alkenyl. R⁵ is a keto-aldehyde or another suitable reagent usable in the synthetic scheme B; preferentially, H, C₁₋₆ alkyl and branched and optionally substituted C₁₋₆ alkyl, C₁₂₋₁₈ alkyl, C₁₂₋₁₈ alkenyl, aryl and substituted aryl, benzyl and substituted benzyl.

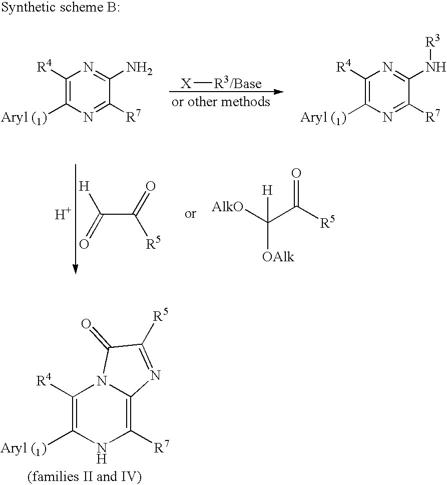

R⁴, R⁵ have the same definition as in formula II

R⁷ is Aryl (2) or H (see scheme A)

R³ has the same definition as in formula II

Preferred embodiments of the compound according to the invention are defined in claims 2-8.

The invention relates to four structural related families of compounds, namely 2-amino-3,5-diaryl-1,4-pyrazine derivatives (formula I),

Formula I:

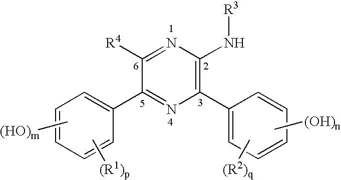

wherein:

n and m are independently 0, 1, 2, 3, 4 or 5;

p and q are independently 0, 1, 2, 3, 4 or 5;

R¹ and R² are independently H, C₁₋₆ alkyl, C₁₂₋₁₈ alkyl, C₁₂₋₁₈ alkenyl, C₁₋₆ oxyalkyl, C₁₂₋₁₈ oxyalkyl or C₁₂₋₁₈ oxyalkenyl, fluoro, cyano, ketone, aldehyde, sulfone, nitro or any electron withdrawing group;

R³ is H or the radical of an alkylating reagent; preferably benzyl and substituted benzyl, C₁₋₆ alkyl and branched and optionally substituted C₁₋₆ alkyl with carboxyl functions and derived functions, C₁₂₋₁₈ alkyl, C₁₂₋₁₈ alkenyl;

R⁴ is H, NH₂ or NHR³,

the derived 6,8-disubstituted imidazolopyrazinone compounds (formula II),

Formula II:

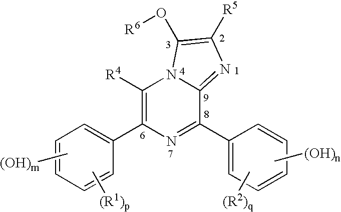

wherein:

n, m, p, q; R¹, R², R⁴ have the same definition as in formula I;

R⁵ is H or the radical of a keto-aldehyde reagent; preferably, H, C₁₋₆ alkyl and branched and optionally substituted C₁₋₆ alkyl, C₁₂₋₁₈ alkyl, C₁₂₋₁₈ alkenyl, aryl and substituted aryl benzyl and substituted benzyl;

R⁶ is H, SO₃⁻M⁺, COMe or glucoronic conjugate,

2-amino-5-aryl-1,4-pyrazine derivatives (formula III)

Formula III:

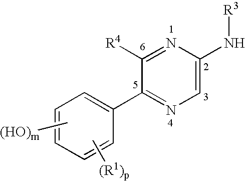

wherein:

m, p, R¹, R³, R⁴ have the same definition as in formula I,

and the derived 6-monosubstituted imidazolopyrazinone compounds (formula IV)

Formula IV:

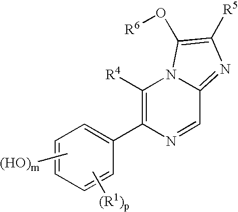

wherein:

m, p, R¹, R⁴, R⁵ have the same definition as in formula II

R⁶ is H₁SO₃⁻M⁺, COMe.

Some of the compounds of family III are intermediates in the synthesis of compounds of family I, as pictured in the synthetic scheme A.

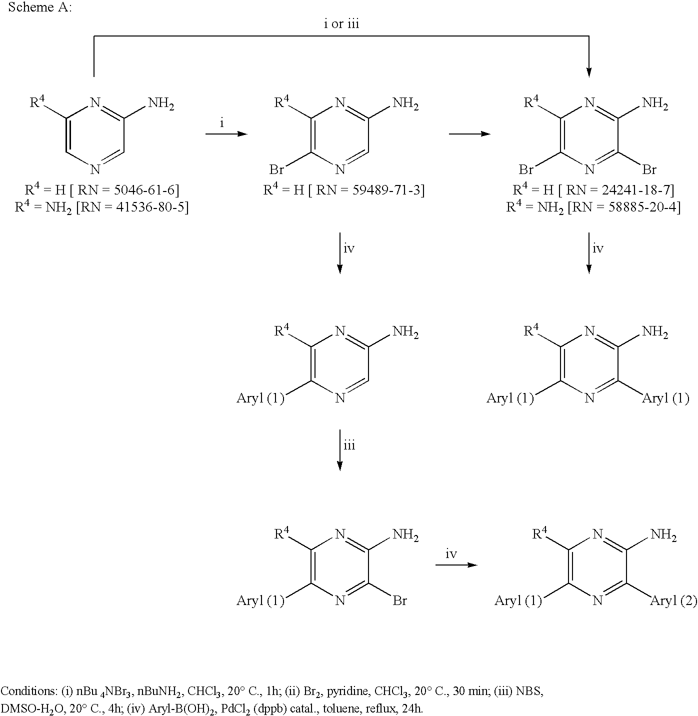

Compounds of families II and IV are synthesized, respectively, from families I and III wherein R³ is H as shown in the synthetic scheme B.

Preferentially, in families I and II, one of the two aryl substituents, or both, are phenol-(ortho, meta, para), or catechol groups. These aromatic groups (one or both) can be substituted with alkyl (or alkoxyl) chains for increasing the lipophilicity of the molecules (R¹ and/or R²). The same effect can be obtained with appropriate R³ or R⁵ substituents.

Preferentially, in families III and IV, the aryl substituent is a catechol group, which can be substituted with alkyl (or alkoxyl) chains as above.

Stabilized forms of these families make part of the invention, i.e. enol derivatives of families II and IV such as described in Inoue S. et al., Tetrahed. Lett. 31 (1977): 2685-2688 and Chem. Lett. 1987: 417-418 included herein by reference, and masked phenol derivatives of families I, II, III and IV, including compounds masked with groups removable in biological fluids (prodrugs). The invention also includes the salts formed by the aminopyrazine and imidazolopyrazinone compounds in the presence of acids.

The invention is also related to the use of said compounds as such, or in compositions.

In the food industry, these present compounds are useful in the protection of raw and processed food and beverages against oxidation.

In the polymer industry, these present compounds are useful for example in food packaging materials, paints, in order to slow down aging processes linked to light, oxygen, and so increase the lifetime of these products.

Further, these compounds according to the invention can be used in lips and skin protection creams and lotions, UV-screens, anti-aging creams.

The present compounds are also useful in human and veterinary medicines for the prevention and the treatment of diseases linked to oxidative damages, such as inflammatory-immune diseases (e.g. rheumatoid arthritis, glomerulonephritis, autoimmune diseases, vasculitis, joint diseases, tendinitis, disc disease, spondylosis), ischemia-reperfusion injury (e.g. stroke, myocardial infarction, organ transplantation, cancer, aging, alcoholism, red blood cell defects, iron overload (e.g. nutritional deficiencies, Kwashiorkor, thalassemia, dietary iron overload, idiopathic hemochromatosis, kidney (e.g. metal-ion mediated nephrotoxicity, aminoglycoside nephrotoxicity, autoimmune nephrotic syndromes), gastrointestinal tract (e.g. oral iron poisoning, endotoxin liver injury, free fatty-acid induced pancreatitis, nonsteroidal antiinflammatory drug induced gastrointestinal tract lesions), heart and cardiovascular system (e.g. atherosclerosis, adriamycin cardiotoxicity, Keshan disease, alcoholic cardiomyopathy, eye (e.g. photic retinopathy, ocular hemorrhage, cataractogenesis, degenerative retinal damage), brain (e.g. neurotoxicity, allergic encephalomyelitis, potentiation of traumatic injury, hypertensive cerebrovascular injury, vitamin E deficiency, Alzheimer's disease, Parkinson's disease), amyelotrophic lateral sclerosis, and age-related macular degeneration. Lotions containing the compounds can be applied topically for local action, injected, or administered orally.

Many of these compounds according to the invention are fluorescent and their reaction with reactive oxygen species (ROS) are accompanied by changes in the fluorescence spectra. Also, imidazolopyrazinones are chemiluminescent compounds which could be used for the detection and quantification of ROS in chemical and biological processes. Long chain alkyl-substituted aminopyrazines and imidazolopyrazinones could be used for the detection of ROS in biological membranes. Also, aminopyrazines and imidazolopyrazinones could be used as substrates for peroxidases and so serve in the detection of these enzymes or their peroxide cofactors. They could also be useful as substrates for coelenterazine-based luciferases and photoproteins and serve in the detection and the quantification of these enzymes, such as in gene reporting studies and peroxidase-linked antibodies for the detection of other compounds.

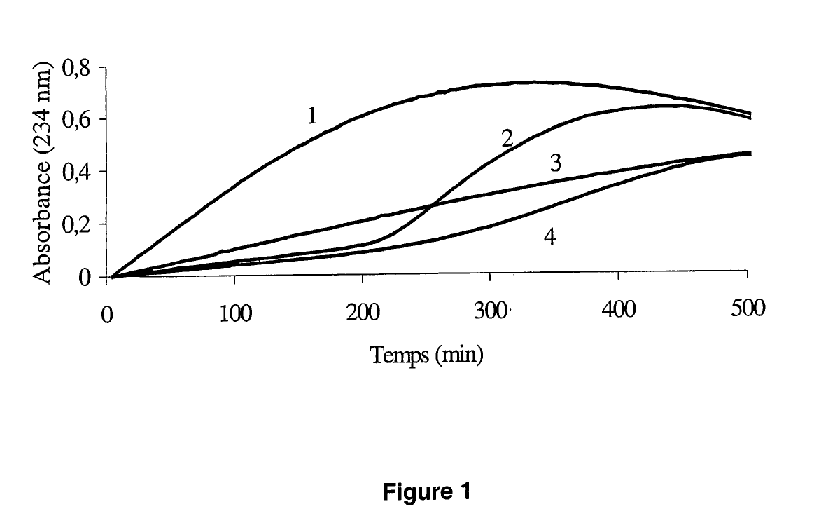

In a preferred embodiment the synthetic derivatives of 2-amino-1,4-pyrazine of family I, characterized by two aryl substituents in positions C-3 and C-5, were endowed with unexpectedly high antioxidative properties (FIG. 1). FIG. 1 shows the inhibition of AAPH-induced peroxidation of linoleic acid by aminopyrazines. AAPH 4 mM was added to a micellar solution of linoleate (0.16 mM). The rate of conjugated dienes formation was measured at 234 nm at 37° C. All aminopyrazines were tested at 5 μM. Curve 1 shows the MPH; curve 2 shows the vitamine E (α-tocopherol); curve 3 shows the CLM and curve 4 shows the JFC38.

Preferably at least one of two aryl substituents was functionalized with one hydroxyl group, preferentially in the para position. These monocyclic compounds were prepared according to the synthetic scheme A.

The symmetrically substituted derivatives (same aryl substituents in positions C-3 and C-5) were prepared from 2-amino-3,4-dibromo-1,4-pyrazine by a double Suzuki-type coupling reaction using the appropriate functionalized arylboronic acid derivatives. The simple Suzuki-like coupling reaction has been used in the total synthesis of CLM (K. Jones, M. Keenan, F. Hibbert, Synlett, 1996, 509-510).

The unsymmetrically substituted compounds (different aryl substituents in positions C-3 and C-5) were prepared in sequence: (a) 2-amino-5-bromo-1,4-pyrazine was coupled with the first arylboronic acid derivative; (b) the resulting 2-amino-5-aryl(1)-1,4-pyrazine was brominated; (c) the resulting 2-amino-3-bromo-5-aryl(1)-1,4-pyrazine was coupled with the second arylboronic acid derivative to give the 2-amino-3-aryl(2)-5-aryl(1)-1,4-pyrazine.

2,6-Diamino-1,4-pyrazine could be similarly derivatized, by bromination followed by a double coupling with arylboronic acid derivatives, to give 2,6-diamino-3,5-diaryl-1,4-pyrazines. The 2-amino- and 2,6-diamino-3,5-diaryl-1,4-pyrazines can be further transformed by N-alkylation.

The second general structure of family II concerns imidazolopyrazinones derived from 2-amino-3,5-diaryl-1,4-pyrazines, which were obtained by condensation with glyoxal derivatives under acidic conditions.

The third general structure of family III discloses 2-amino-1,4-pyrazines characterized by one aryl substituent in position C-5; preferentially the aryl is a catechol. This third group is endowed with an extremely high antioxidant activity. Some of the compounds of family III are intermediates in the synthesis of compounds of family I. The 2-amino- and 2,6-diamino-3-aryl-1,4-pyrazines can be further transformed by N-alkylation.

The fourth general structure of family IV concerns imidazolopyrazinones derived from 2-amino-3-aryl-1,4-pyrazines, which were obtained by condensation with glyoxal derivatives under acidic conditions.

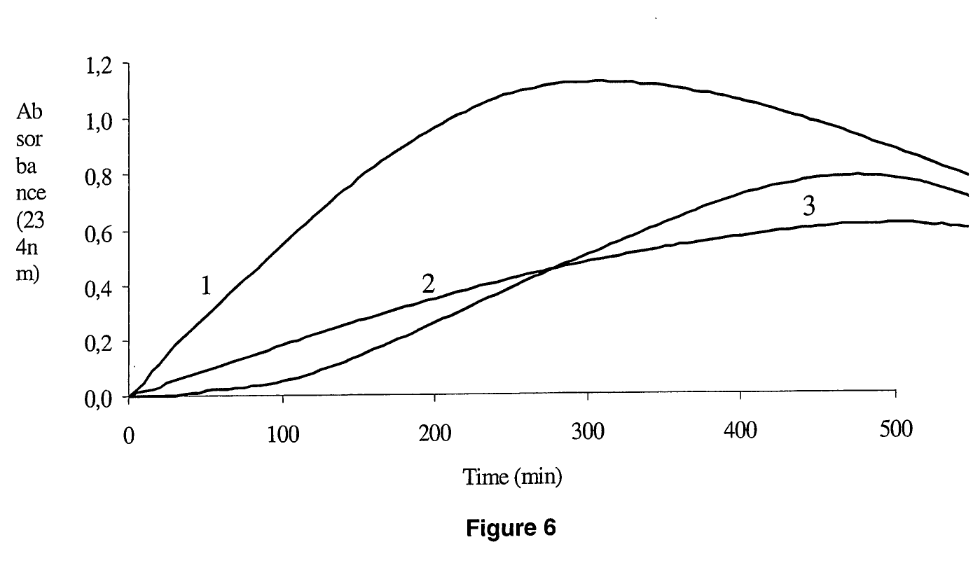

In another aspect the invention is also related to a new method of administrating anti-oxidant compounds via a cascade effect. This cascade effect results in first and second generation anti-oxidant compounds and is able in a further preferred embodiment to generate a third generation of a compound which can have any suitable action, such as a anti-inflammatory action. This “cascade” effect can be explained using mother-daughter compounds. The imidazolopyrazinone antioxidant (mother-compound) (see formula II and formula IV) is upon oxidation able to liberate another antioxidant, namely the corresponding aminopyrazine (daughter-compound) (see formula I and formula is II). This second-generation antioxidant is therefore delivered on the site of action of the first-generation drug (see FIG. 6, with CD51 and CD53), as explained hereunder:

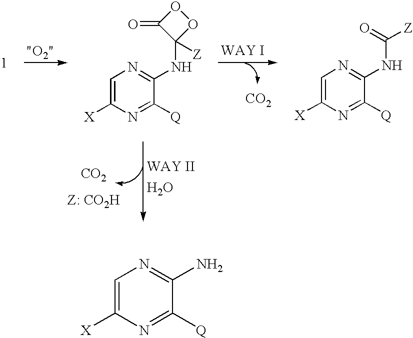

wherein X, Q and Z are all suitable substituents available in the formula's I-V and way I is the bioluminous way and way II shows the cascade antioxydative way. It is also possible that the resulting Z CO₂H is as such or as a precursor another suitable active compound, such as a third antioxidant or an anti-inflammatory agent.

The mother-compound firstly delays the onset of the oxidation process in lipid peroxidation (AAPH-induced) and then reduces the rate of the oxidation, while the daughter-compound only reduces the rate of the oxidation.

### EXAMPLES

1. General Procedure for the Suzuki-Like Coupling Reaction.

A mixture of bis(benzonitrile)palladium(II)dichloride (0.05 eq. from Acros) and 1,4-bis(diphenylphosphino)butane (dppb) (0.06 eq. from Acros) in dry toluene was stirred at room temperature under argon atmosphere for 30 min until a creamy orange slurry of [1,4-bis(diphenylphosphino)butane]-palladium(II)chloride was formed. Amino-bromopyrazine (1 eq.), arylboronic acid (1.1 eq. from Aldrich), ethanol, aqueous sodium carbonate solution (1 M, 1 eq.) and toluene were added to the preformed catalyst and the mixture was heated under reflux for 24 hours. After cooling to room temperature, water was added and the mixture diluted with ethyl acetate. The aqueous phase was separated and extracted twice with ethyl acetate. The combined organic phases were then washed twice with brine, dried (MgSO₄), filtered over celite and concentrated in vacuum. The crude product was purified by column chromatography on silica gel.

2. General Procedure for the Deprotection of Aryl Methyl Ether.

A stirred solution of (4-methoxyphenyl)pyrazine (1 eq.), sodium ethanethiolate (8 eq.) in DMF was heated (under argon atmosphere) at 100° C. during 24 hours. After cooling to room temperature, ethyl acetate and a saturated solution of ammonium chloride were added. The aqueous phase was extracted with ethyl acetate (4×), and the combined organic layers were washed with brine (2×), dried (MgSO₄), filtered and concentrated in vacuum. The crude solid was washed with a solution of ether/ethyl acetate 1:1.

3. General Procedure for the Condensation Reaction with Glyoxal Derivatives.

A mixture of 2-amino-(3),5-(di)arylpyrazine (1 eq.), methyl glyoxal (40 wt % solution in water, 1.5 eq.) and 37% aqueous HCl (3.6 eq.) in ethanol was heated (under argon atmosphere) at 80° C. during 4 hours. After cooling to room temperature, the solution was concentrated in vacuum and the crude solid was successively washed with ethyl acetate and ether to afford the imidazolopyrazinone as the hydrochloride monohydrate.

4. General Procedure for the N-Benzylation Reaction

A mixture of 2-amino-1,4-pyrazine (1 eq.), LiHMDS (1.5 eq.) in dry THF was stirred 1 hour at room temperature. A solution of benzylbromide (1.1 eq.) in dry THF was added dropwise and the reaction was then stirred overnight (total stirring 20 hours). Ethyl acetate was added and the organic layer was washed with 5% aqueous sodium carbonate (2×) and brine (3×). The aqueous phases were re-extracted with ethyl acetate (3×) and the organic layer was dried (MgSO₄), filtered and concentrated in vacuum. The crude product was purified by silica-gel chromatography.

5. 2-Amino-3,5-bis(p-methoxyphenyl)-1,4-pyrazine (CD29) and 2-amino-3,5-bis(p-hydroxyphenyl)-1,4-pyrazine (CD31).

Starting from bis(benzonitrile)palladium(II)dichloride (306.3 mg, 0.10 eq.), 1,4-bis(diphenylphosphino)butane (408.3 mg, 0.12 eq.), 2-amino-3,5-dibromopyrazine (2.02 g, 8 mmol, 1 eq.; prepared by bis-bromination of 2-aminopyrazine), 4-methoxyphenylboronic acid (2.55 g, 2.1 eq.), ethanol (6.8 mL), aqueous sodium carbonate solution (16 mL, 1 M, 2 eq.) and toluene (2×20 mL), 2-amino-3,5-bis(4-methoxyphenyl)-1,4-pyrazine (1.6 g, 66%) was obtained as a yellow solid. This compound does not make part of the invention. It has been prepared previously by Stille coupling (Nakamura, H.; Takeuchi, D.; Murai, A. Synlett 1995, 1227-1228).

m.p. 136.6° C.

Silica-gel chromatography: R_{f}=0.21, EtOAc/cyclohexane 3:5

EA calcd for C₁₈H₁₇N₃O₂ (307.35 g.mol⁻¹): C, 70.34; H, 5.57; N, 13.67. Found: C, 70.16; H, 5.56; N, 13.53.

The protected precursor (pOMe) (1.6 g, 5.21 mmol, 1 eq.) treated with EtSNa (3.50 g, 8 eq.) in DMF (25 ml) gave 2-amino-3,5-bis(4-hydroxyphenyl)-1,4-pyrazine (1.28 g, 88%) as a yellow solid. m.p. 251.1° C.

EA calcd for C₁₆H₁₃N₃O₂.½H₂O (288.3 g.mol⁻¹): C, 66.59; H, 4.85; N, 14.57. Found: C, 66.85; H, 5.56; N, 13.42.

6. 2,6-Diamino-3,5-bis(p-methoxyphenyl)-1,4-pyrazine (JFC26) and 2,6-diamino-3,5-bis(p-hydroxyphenyl)-1,4-pyrazine (JFC28).

Starting from bis(benzonitrile)palladium(II)dichloride (98.65 mg, 0.10 eq.), 1,4-bis(diphenylphosphino)butane (131.57 mg, 0.12 eq.), 2,6-diamino-3,5-dibromopyrazine (689 mg, 2.57 mmol, 1 eq.; prepared by bis-bromination of 2,6-diaminopyrazine), 4-methoxyphenylboronic acid (860 mg, 2.2 eq.), ethanol (2.28 mL), aqueous sodium carbonate solution (5.2 mL, 1 M, 2 eq.) and toluene (2×10 mL), 2,6-diamino-3,5-bis(4-methoxyphenyl)-1,4-pyrazine (678.5 mg, 82%) was obtained as a yellow solid.

m.p. 152.3° C.

Silica-gel chromatography: R_{f}=0.11, EtOAc/cyclohexane 3:5.

EA calcd for C₁₈H₁₈N₄O₂ (322.36 g.mol⁻¹): C, 67.10; H, 5.60; N, 17.40. Found: C, 66.94; H, 5.45; N, 17.21.

2,6-Diamino-3,5-(4-methoxyphenyl)-1,4-pyrazine (360 mg, 1.12 mmol, 1 eq.) treated with sodium ethanethiolate (790 mg, 8 eq.) in DMF (6 mL) gave, after recrystallization from chloroform, 2,6-diamino-3,5-bis(4-hydroxyphenyl)-1,4-pyrazine (236 mg, 72%) as a red solid.

m.p. 89° C.

EA calcd for C₁₆H₁₄N₄O₂ (312.23 g.mol⁻¹): C, 61.48; H, 5.12; N, 17.93. Found: C, 61.13; H, 5.12; N, 16.17.

7. 2-Amino-3-phenyl-5-(p-methoxyphenyl)-1,4-pyrazine (CD48) and 2-amino-3-phenyl-5-(p-hydroxyphenyl)-1,4-pyrazine (CD51).

Starting from bis(benzonitrile)palladium(II)dichloride (154.7 mg, 0.05 eq.), 1,4-bis(diphenylphosphino)butane (206.3 mg, 0.06 eq.), 2-amino-3-bromo-5-(4-methoxyphenyl)pyrazine (2.26 g, 8.06 mmol, 1 eq.; prepared by bromination of 2-amino-5-(4-methoxyphenyl) pyrazine), phenylboronic acid (1.08 g, 1.1 eq.), ethanol (3.5 mL), aqueous sodium carbonate solution (8.1 mL, 1M, 1 eq.) and toluene (2×15 mL), 2-amino-3-phenyl-5-(4-methoxyphenyl)-1,4-pyrazine (1.95 mg, 87%) was obtained as a yellow solid.

m.p. 124.9° C.

Silica-gel chromatography: R_{f}=0.31, EtOAc/cyclohexane 3:5.

EA calcd for C₁₇H₁₅N₃₀ (277.32 g.mol⁻¹): C, 73.63; H, 5.45; N, 15.15. Found: C 73.25, H 5.38, N, 15.01.

2-Amino-3-phenyl-5-(4-methoxyphenyl)-1,4-pyrazine (1.92 g, 6.92 mmol, 1 eq.) treated with sodium ethanethiolate (2.33 g, 4 eq.) in DMF (15 mL) gave 2-amino-3-phenyl-5-(4-hydroxyphenyl)-1,4-pyrazine (1.25 g, 69%) as a yellow solid.

m.p. 200.3° C.

EA calcd for C₁₆H₁₃N₃₀ (263.19 g.mol⁻¹): C, 72.99; H, 4.98; N, 15.96. Found: C, 72.33; H, 5.11; N, 15.55.

8. 2-Amino-3-(p-methoxyphenyl)-5-phenyl-1,4-pyrazine (CD45) and 2-amino-3-(p-hydroxyphenyl)-5-phenyl-1,4-pyrazine (CD46).

Starting from bis(benzonitrile)palladium(II)dichloride (158.1 mg, 0.05 eq.), 1,4-bis(diphenylphosphino)butane (210.9 mg, 0.06 eq.), 2-amino-3-bromo-5-phenylpyrazine (2.06 g, 8.24 mmol, 1 eq.; prepared by bromination of 2-amino-5-phenylpyrazine), 4-methoxyphenylboronic acid (1.31 g, 1.1 eq.), ethanol (3.5 mL), aqueous sodium carbonate solution (8.3 mL, 1M, 1 eq.) and toluene (2×20 mL), 2-amino-3-(4-methoxyphenyl)-5-phenyl-1,4-pyrazine (1.926 mg, 84%) was obtained as a yellow solid.

m.p. 148.6° C.

Silica-gel chromatography: Rf=0.32, EtOAc/cyclohexane 3:5.

EA calcd for C₁₇H₁₅N₃₀ (277.32 g.mol⁻¹): C, 73.63; H, 5.45; N, 15.15. Found: C, 73.03; H, 5.37; N, 14.98.

2-Amino-3-(4-methoxyphenyl)-5-phenyl-1,4-pyrazine (1.92 g, 6.92 mmol, 1 eq.) treated with sodium ethanethiolate (2.33 g, 4 eq.) in DMF (16 mL) gave 2-amino-3-(4-hydroxyphenyl)-5-phenyl-1,4-pyrazine (1.24 mg, 68%) as a yellow solid.

m.p. 222.3° C.

EA calcd for C₁₆H₁₃N₃₀ (263.29 g.mol⁻¹): C, 72.99; H, 4.98; N, 15.96. Found: C, 72.45; H, 5.02; N, 15.64.

9. 2,6-Bis(1′-ethoxycarbonyl-ethylamino)-3,5-bis(p-hydroxyphenyl)-1,4-pyrazine (JFC38).

A mixture of 2,6-diamino-3,5-bis(4-hydroxyphenyl)-1,4-pyrazine (165 mg, 0.56 mmol, 1 eq.), methyl glyoxal (200 μL, 40 wt % solution in water, 2.2 eq.) and 37% aqueous HCl (170 μL, 3.6 eq.) in ethanol (4.5 mL) was heated (under argon atmosphere) at 80° C. during 4 hours. After cooling to room temperature, the solution was concentrated in vacuum and the crude solid was washed with ether to afford the 2,6-bis(1-ethoxycarbonyl-ethylamino)-3,5-bis(4-hydroxyphenyl)-1,4-pyrazine dihydrochloride (231 mg, 73%), as a red solid.

m.p. 175° C. (dec.).

m/z (FAB+Q1 MS) 495 ((M+H)^{+).}

EA calcd for C₂₆H₃₂Cl₂N₄O₆ (567.54 g.mol⁻¹): C 53.03, H 5.68, Cl 12.50, N 9.87. Found: C 55.16, H 5.62, Cl 11.02, N 9.93.

10. 2-Methyl-6,8-bis(p-hydroxyphenyl)-3,7-dihydroimidazolo[1,2-a]pyrazin-3-one (CD43).

Starting from 2-amino-3,5-bis(4-hydroxyphenyl)-1,4-pyrazine (600 mg, 2.15 mmol, 1 eq.), methyl glyoxal (40 wt % solution in water, 0.5 mL, 1.5 eq.) and 37% aqueous HCl (0.62 mL, 3.6 eq.) in ethanol (20 mL), the hydrochloride monohydrate 2-methyl-6,8-bis(4-hydroxyphenyl)-3,7-dihydroimidazolo[1,2-a]pyrazin-3-one (730.8 mg, 84%) was obtained as a red solid.

m.p. 168.30 C.

m/z (FAB+Q1 MS) 334 ((M+H)⁺), 306 ((M+H—CO)⁺), 291 ((M+H+CH₃)⁺).

EA calcd for C₁₉H₂₀ClN₃O₅ (405.82 g.mol⁻¹) C 56.18, H 4.93, Cl 8.74, N 10.34. Found: C 55.36, H 5.08, Cl 9.80, N 9.34.

11. 2-Methyl-6-(p-hydroxyphenyl)-8-phenyl-3,7-dihydroimidazolo[1,2-a]pyrazin-3-one (CD53).

Starting from 2-amino-3-phenyl-5-(4-hydroxyphenyl)-1,4-pyrazine (450.2 mg, 1.71 mmol, 1 eq.), methyl glyoxal (40 wt % solution in water, 0.40 mL, 1.5 eq.) and 37% aqueous HCl (0.51 mL, 3.6 eq.) in ethanol (7 mL), the hydrochloride monohydrate 2-methyl-6-(p-hydroxyphenyl)-8-phenyl-3,7-dihydroimidazolo[1,2-a]pyrazin-3-one (500 mg, 79%) was obtained as a yellow solid.

m.p. 1800 C (dec.).

m/z (FAB+Q1 MS) 318 ((M+H)⁺), 290 ((M+H-CO)⁺), 275 ((290-CH₃)⁺), 249 ((290-CH₃CN)⁺).

EA calcd for C₁₉H₁₈ClN₃O₃ (371.81 g.mol⁻¹) C 61.40, H 4.90, Cl 9.50, N 11.30. Found: C 60.55, H 5.03, Cl 10.05, N 10.76.

12. 2-Methyl-6-phenyl-8-(p-hydroxyphenyl)-3,7-dihydroimidazolo[1,2-a]pyrazin-3-one (CD52).

Starting from 2-amino-3-(4-hydroxyphenyl)-5-phenyl-1,4-pyrazine (279 mg, 1.06 mmol, 1 eq.), methyl glyoxal (40 wt % solution in water, 0.25 mL, 1.5 eq.) and 37% aqueous HCl (0.32 mL, 3.6 eq.) in ethanol (4 mL), the hydrochloride monohydrate 2-methyl-6-phenyl-8-(p-hydroxyphenyl)-3,7-dihydroimidazolo[1,2-a]pyrazin-3-one (324 mg, 82%) was obtained as a yellow solid.

m.p. 99.5° C. (dec.).

m/z (FAB+Q1 MS) 318 ((M+H)⁺), 290 ((M+H—CO)⁺), 275 ((290-CH₃)⁺), 249 ((290-CH₃CN)⁺).

EA calcd for C₁₉H₁₈ClN₃O₃ (371.81 g.mol⁻¹): C 61.40, H 4.90, Cl 9.50, N 11.30. Found: C 60.72, H 5.06, Cl 10.11, N 10.67.

13. 2-Amino-5-(3,4-dimethoxyphenyl)-1,4-pyrazine (JFC48) and 2-Amino-5-(3,4-dihydroxyphenyl)-1,4-pyrazine (JFC58).

Starting from bis(benzonitrile)palladium(II)dichloride (96 mg, 0.05 eq.), 1,4-bis (diphenylphosphino)butane (128 mg, 0.06 eq.), 2-amino-5-bromopyrazine (869 mg, 5 mmol, 1 eq.; prepared by bromination of 2-aminopyrazine), 3,4-dimethoxyphenylboronic acid (1.0 g, 1.1 eq.), ethanol (2.2. mL), aqueous sodium carbonate solution (5 mL, 1M, 1 eq.) and toluene (2×12 mL), 2-Amino-5-(3,4-dimethoxyphenyl)-1,4-pyrazine (930 mg, 80%) was obtained as a yellow solid.

m.p. 191° C.

silica-gel chromatography: Rf=0.16, EtOAc/cyclohexane 3:5

m/z (El+Q1 MS) 231 (M^{+.}), 216 ((M-CH₃)^{+.}).

2-Amino-5-(3,4-dimethoxyphenyl)-1,4-pyrazine (700 mg, 3.03 mmol, 1 eq.) treated with sodium ethanethiolate (2.04 g, 8 eq.) in DMF (16 mL) gave, after recrystallization from chloroform, 2-Amino-5-(3,4-dihydroxyphenyl)-1,4-pyrazine (180 mg, 29%) as a red solid.

m.p. 136° C. (dec.)

m/z (FAB+Q1 MS) 204 ((M+H)⁺).

14. 2-(N-benzylamino)-5-(p-methoxyphenyl)-1,4-pyrazine (JFC55) and 2-(N-benzylamino)-5-(p-hydroxyphenyl)-1,4-pyrazine (JFC71).

Starting from 2-amino-5-(p-methoxyphenyl)-1,4-pyrazine (500 mg, 1 eq.), LiHMDS (900 mg, 1.5 eq.), Benzylbromide (467 mg, 1.1 eq.) and THF (13+5 mL), 2-(N-benzylamino)-5-(p-methoxyphenyl)-1,4-pyrazine (303 mg, 42%) was obtained as a yellow solid.

m.p. 153° C.

silica-gel chromatography: Rf=0.47, EtOAc/cyclohexane 3:5

EA calcd for C₁₈H₁₇N₃O (291.35 g.mol⁻¹): C 74.20; H 5.90; N 14.40. Found C 73.40; H 5.97; N 13.86.

The protected precursor (p-OMe) (289 mg, 0.99 mmol, 1 eq.) treated with EtSNa (333 mg, 4 eq.) in DMF (5 mL) gave 2-(N-benzylamino)-5-(p-hydroxyphenyl)-1,4-pyrazine (75 mg, 27%) as a yellow solid.

m.p. 160° C.

m/z (El+Q1 MS) 277 (M^{+.})

15. 2-(N-benzylamino)-3,5-bis-(p-methoxyphenyl)-1,4-pyrazine (JFC72) and 2-(N-benzylamino)-3,5-bis-(p-hydroxyphenyl)-1,4-pyrazine (JFC73).

Starting from 2-amino-3,5-bis-(p-methoxyphenyl)-1,4-pyrazine (500 mg, 1 eq.), LiHMDS (592 mg, 1.5 eq.), Benzylbromide (306 mg, 1.1 eq.) and THF (7+5 mL), 2-(N-benzylamino)-3,5-bis-(p-methoxyphenyl)-1,4-pyrazine (404 mg, 62%) was obtained as a yellow solid.

m.p. 101-102° C.

silica-gel chromatography: Rf=0.44, EtOAc/cyclohexane 3:5

EA calcd for C₂₅H₂₃N₃O₂ (397.35 g.mol⁻¹): C 75.50; H 5.79; O 10.57. Found C 75.46; H 5.78; N 10.41.

The protected precursor (p-OMe) (200 mg, 0.5 mmol, 1 eq.) treated with EtSNa (338 mg, 8 eq.) in DMF (5 mL) gave 2-(N-benzylamino)-3,5-bis-(p-hydroxyphenyl)-1,4-pyrazine (147 mg, 79%) as a yellow solid.

m.p 90.5° C.

m/z (El+Q1 MS) 369 (M^{+.}).

16. 2-Methyl-8-(3,4-dihydroxyphenyl)-3,7-dihydroimidazolo[1,2-4]pyrazin-3-one (JFC66)

Starting from 2-Amino-5-(3,4-dihydroxyphenyl)-1,4-pyrazine (54 mg, 0.26 mmol, 1 eq.), methyl glyoxal (40 wt % solution in water, 0.06 mL, 1.5 eq.) and 37% aqueous HCl (0.08 mL, 3.6 eq.) in ethanol (1.3 mL), the hydrochloride monohydrate 2-Methyl-B-(3,4-dihydroxyphenyl)-3,7-dihydroimidazolo[1,2-4]pyrazin-3-one (50 mg, 61%) was obtained as a red solid.

m.p. 162.2° C. (dec.).

m/z (FAB+Q1 MS) 258 ((M+H)⁺).

17. Other examples are 2-amino-3-(3,4-dihydroxyphenyl)-5-(4-hydroxyphenyl)-1,4-pyrazine (JFC54)

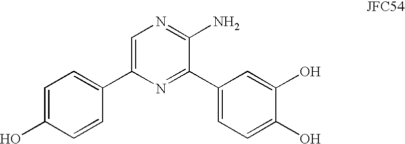

and

2-methyl-6-(3,4-dihydroxyphenyl)-8-(4-hydroxyphenyl)-3,7-dihydro-imidazolo[1,2-a]pyrazin-3-one (JFC81)

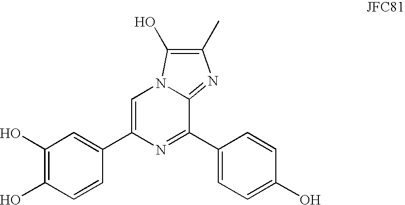

The structure of all these compounds is disclosed hereunder.

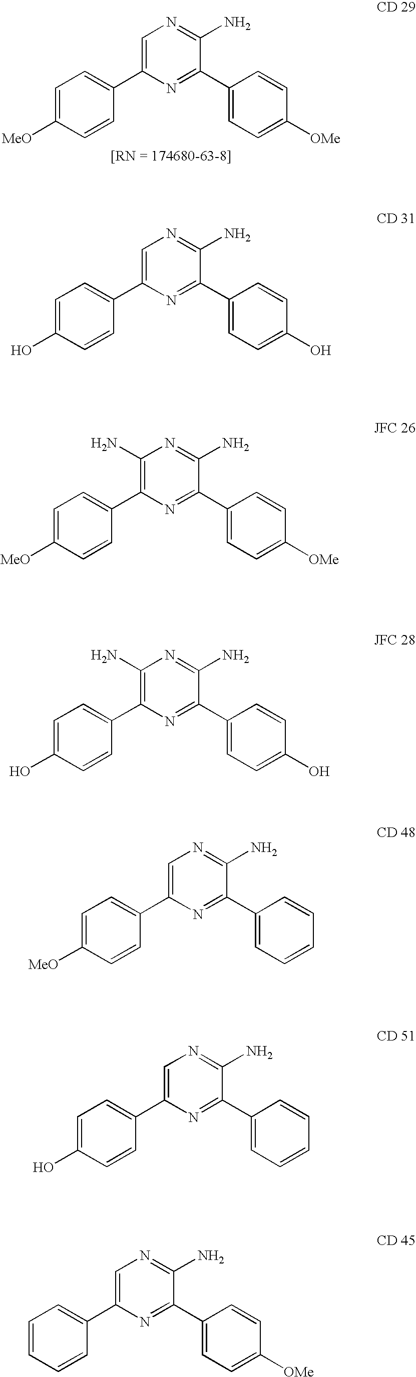

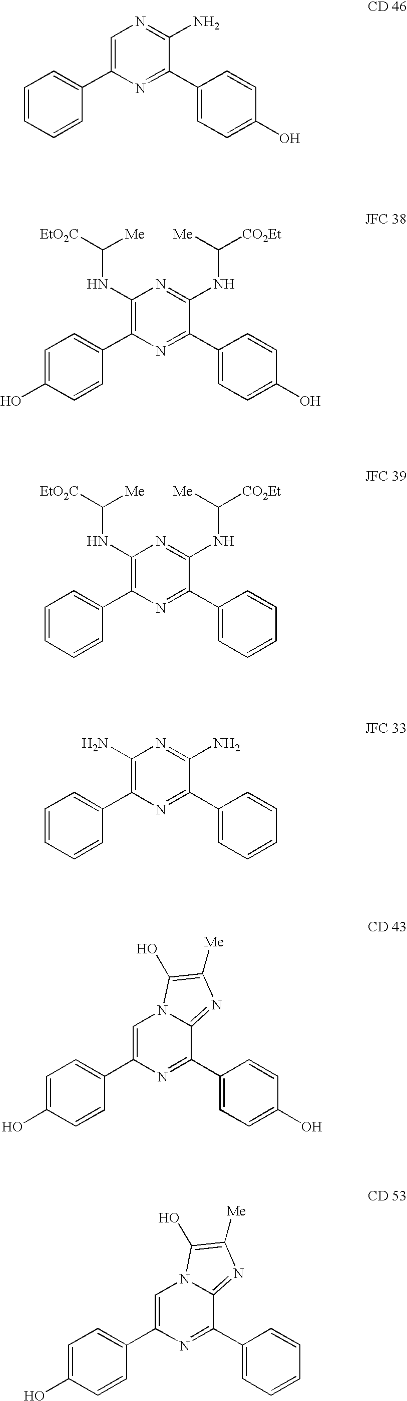

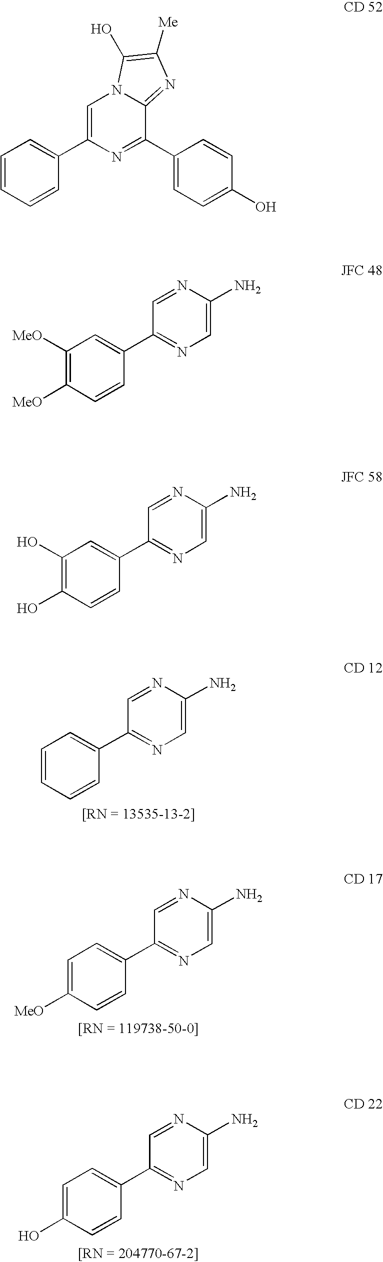

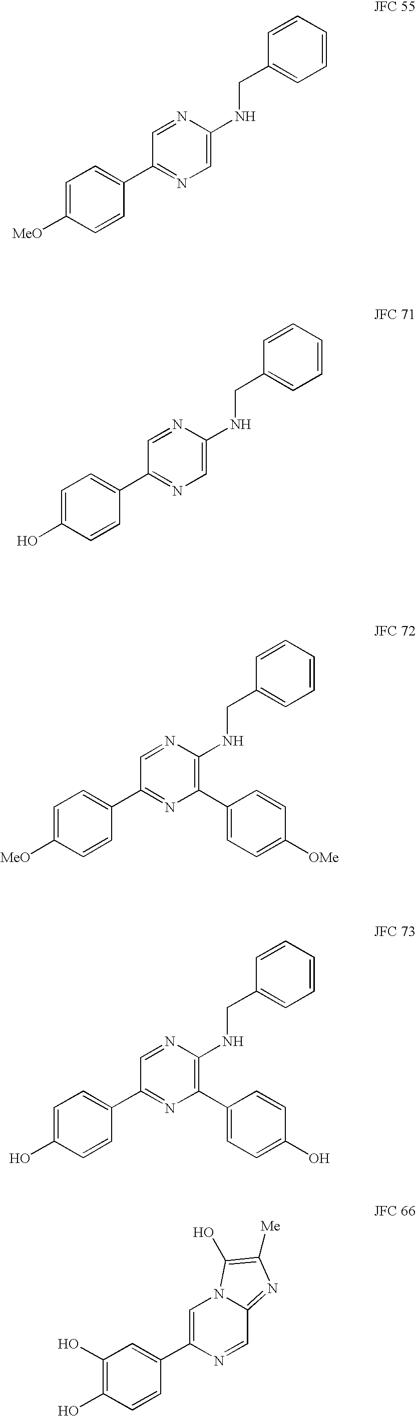

Possible substitutions on the aryl substituents are listed in table 1 and table 4.

Experiments

1. inhibition of lipid peroxidation.

The ability of the synthesized compounds to inhibit lipid peroxidation has been tested on AAPH-induced oxidation of linoleate. Briefly, a micellar solution of linoleate (0.16 mM) is incubated at 37° C. with 4 mM free radical generator AAPH (2,2′-azobis-2-methyl-propionamidine hydrochloride) in a microplate-based spectrophotometer. The production of conjugated dienes by the peroxidation of linoleate is monitored continuously at 234 nm. Antioxidants can both delay the onset of the oxidation process and reduce the rate of the oxidation.

Results

Table 2:

2-Aminopyrazines possessing two aryl substituents, one of them being a p-hydroxyphenyl in ortho-or para-position with respect to the amino group, are endowed with antioxidative properties. However, the p-hydroxyphenyl conferred more activity when located at position 5 (CD51) than at position 3 (CD46). The presence of phydroxyphenyl groups at both positions 3 and 5 (CD31) produced a very active compound. Analogue lacking the free phenol groups (CD29) showed reduced activities.

Table 2 shows the inhibition of lipid peroxidation by aminopyrazines. The amount of conjugated dienes formed by the peroxidative process evaluated by the absorption at 234 nm after 150 min at 37° C. All aminopyrazines were tested at 10 μM.

TABLE 2

| Treatment | A₂₃₄ |
|---|---|
| AAPH alone | 0.37 |
| +CD31 | 0.08 |
| +CD51 | 0.12 |
| +CD46 | 0.19 |
| +CD29 | 0.37 |

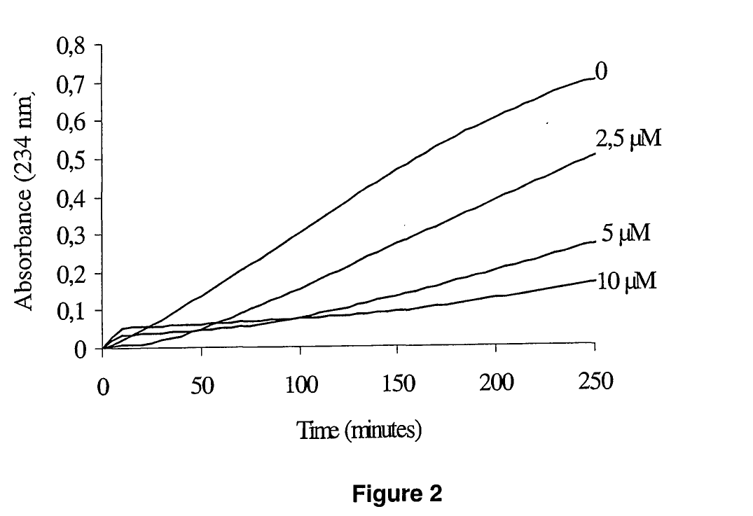

Corresponding imidazolopyrazinones (e.g. CD43) combined the properties of both the imidazolopyrazinones (delay of the onset of peroxidation) and the aminopyrazines (lower rate of oxidation after onset). FIG. 2 shows inhibition of AAPH-induced peroxidation of linoleic acid by increasing concentrations of CD43. The rate of conjugated dienes formation was measured at 234 nm. The procedure is identical as in FIG. 1.

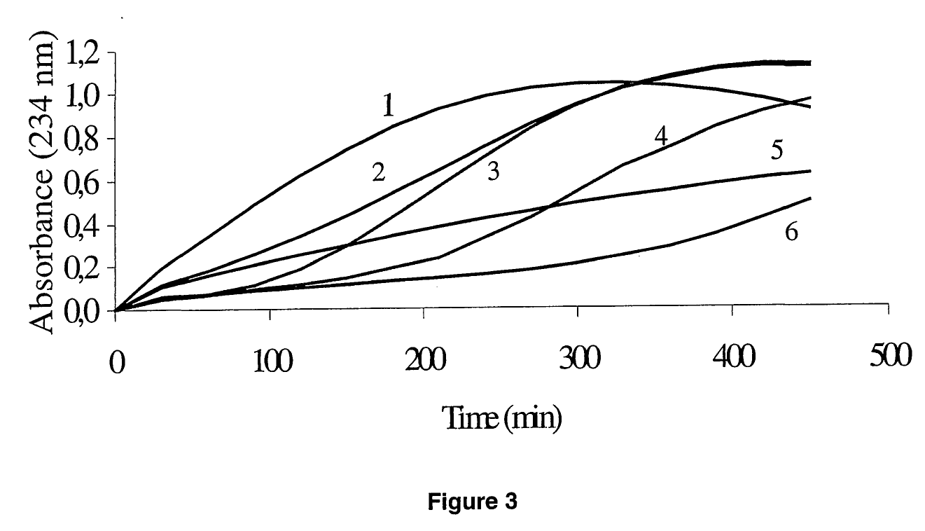

Addition of a second amino group at position 6 maintains the antioxidative activity of the aryl-substituted pyrazines. However, it does not improve the activity (JFC28 versus CD31) as one may expected since two phydroxyphenyl groups are present on the pyrazine ring. However, the presence of two amino groups renders phenyl substituted pyrazines active (JFC 33 and JFC 39). The grafting of an alkyl chain on both amino groups makes the bis-phydroxyphenyl compound very active (JFC 38). FIG. 3 shows the inhibition of AAPH-induced peroxidation of linoleic acid by aminopyrazines. The procedure is identical as in FIG. 1. The rate of conjugated dienes formation was measured at 234 nm. All aminopyrazines were tested at 5 μM. Curve 1 shows the AAPH; curve 2 shows the JFC33; curve 3 shows the JFC39; curve 4 shows the JFC28; curve 5 shows the CD31 and curve 6 shows the JFC38.

The imidazolopyrazynones CD10 and CD11 delayed the onset of the lipid peroxidation with a similar efficiency. The induction period induced by both molecules increased with their concentrations. Their antioxidant activity is similar to that observed with Trolox, a water-soluble vitamine E analogue. FIG. 5 shows the inhibition period (lag time) observed in the oxidation of AAPH-induced linoleic acid peroxidation in the presence of various concentrations of CD10 and CD11.

FIG. 6 shows the inhibition of AAPH-induced peroxidation of linoleic acid by aminopyrazines. The procedure is identical as in FIG. 1. The rate of conjugated dienes formation was measured at 234 nm. All molecules were tested at 5 μM. Curve 1 shows the AAPH; curve 2 shows the CD51 and curve 3 shows the CD53.

In the examples illustrated in FIG. 6, the mother-compound CD53 firstly delays the onset of the oxidation process, while the daughter-compound CD51, only reduces the rate of the oxidation.

2. Protective effect against UVB

Human keratinocytes cells (HaCaT) were cultured in 96-well microplates. Tested compounds were solubilized in phosphate buffered saline (PBS) and applied to cells 30 minutes prior to the irradiation with UVB in a BIOSUN irradiation system (Vilbert-Lourmat). PBS was then replaced by the culture medium containing the tested compounds, and incubated for a further 24 hours before measuring the percentage of lactate dehydrogenase (LDH) released into the cell culture supernatant.

Results

Table 3:

Results obtained on UVB-treated keratinocytes confirmed the protective effect of 3,5-diaryl-2-amino-1,4-pyrazines. Some compounds such as CD46, which although showing lower efficiency than CD31 in inhibiting lipid peroxidation in vitro, very efficiently reduced the cytotoxicity of UVB. None of these compounds were cytotoxic for the cells.

Table 3 shows the protection by aminopyrazines CD31, CD46 and CD51 of HaCaT cells against UVB-induced cytotoxicity. Keratinocytes were pre-incubated with aminopyrazines (50 μM) for 30 min and then irradiated with UVB at 200 mJ/cm² in the absence of the antioxidant. The cells were then incubated for a further 24 hours in the culture medium containing the tested compounds before measuring the percentage of lactate dehydrogenase (LDH) released into the cell culture supernatant.

TABLE 3

Percentage Mortality (LDH assay)

| Treatment | Unirradiated cells | UVB-irradiated cells |
|---|---|---|
| Control | 0 | 14.1 |
| CD31 | 0 | 0 |
| CD46 | 0 | 0 |
| CD51 | 0 | 7.3 |

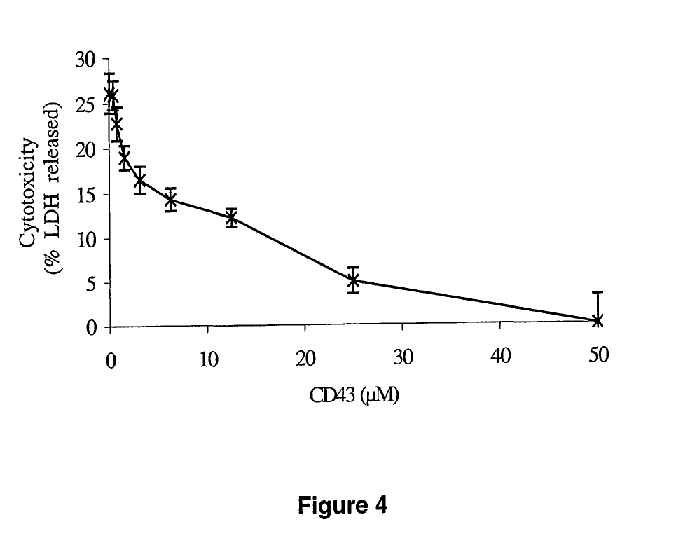

Corresponding imidazolopyrazinones (e.g. CD43) also very efficiently protect cells against UVB-induced mortality; Controls indicated that they showed no toxicity for cells. FIG. 4 shows the protection by imidazolopyrazine CD43 of HaCaT cells against UVB-induced cytotoxicity. Keratinocytes were pre-incubated with increasing concentrations of CD43 for 30 min and then irradiated with UVB at 200 mJ/cm². The cells were incubated for a further 24 hours before measuring the percentage of lactate dehydrogenase (LDH) released into the cell culture supernatant.

TABLE 1
representative aryl substitutions

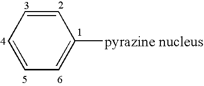

| Position | 2 | 3 | 4 | 5 | 6 |
|---|---|---|---|---|---|
| a | H | H | H | H | H |
| b | OH | H | H | H | H |
| c | H | OH | H | H | H |
| d | H | H | OH | H | H |
| e | H | OH | OH | H | H |
| f | OH | H | OH | H | H |
| g | OH | H | H | CH₃ | H |
| h | OH | H | H | C₁₆H₃₃ | H |
| i | CH₃ | H | H | H | H |
| j | C₁₆H₃₃ | H | H | H | H |
| k | OCH₃ | H | H | H | H |
| l | OC₁₆H₃₃ | H | H | H | H |
| m | H | CH₃ | H | H | H |
| n | H | C₁₆H₃₃ | H | H | H |
| o | H | OCH₃ | H | H | H |
| p | H | OC₁₆H₃₃ | H | H | H |
| q | H | H | CH₃ | H | H |
| r | H | H | C₁₆H₃₃ | H | H |
| s | H | H | OCH₃ | H | H |
| t | H | H | OC₁₆H₃₃ | H | H |

TABLE 4
representative aryl substitutions

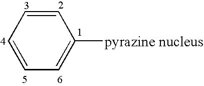

| position | 2 | 3 | 4 | 5 | 6 |
|---|---|---|---|---|---|
| a | H | H | F | H | H |
| b | F | H | F | H | H |
| c | F | H | H | H | F |
| d | F | F | F | F | F |
| e | CF₃ | H | H | H | H |
| f | H | NO₂ | H | H | H |
| g | H | CN | H | H | H |
| h | H | H | COMe | H | H |
| i | COMe | H | H | H | H |
| j | H | H | CO₂H | H | H |
| k | H | CO₂H | H | H | H |

### REFERENCES

Devillers, I. et al. J. Chem. Soc. Perkin, Trans 2,1999,1481-1487.

Hirano, T. et al. Tetrahedron, 1993, 49, 9267-9276.

Ohmiya, Y. et al. Chem. Lett., 1993, 2149-52.

Alcaide, B. et al. J. Org. Chem. 1990, 55, 3143-7.

Alcaide, B. et al. J. Org. Chem. 1989, 54, 5763-8.

Yamaguchi, l. Biochem. J. 1975,151,9-15.

1. An aryl substituted pyrazine compound of the general formula I, II, III or IV with the exception of a) 2-amino-3,5-bis(p-methoxyphenyl)-1,4-pyrazine (CD29), 2-amino-5-phenyl-1,4-pyrazine (CD12), 2-amino-5-(4-methoxyphenyl)-1,4-pyrazine (CD17), 2-amino-5-(4-hydroxyphenyl)-1,4-pyrazine (CD22), 5-phenyl-2-methylamino-1,4-pyrazine and 2-amino-3,5-bis-phenyl-1,4-pyrazine and of b) their corresponding imidazolopyrazinone compounds, 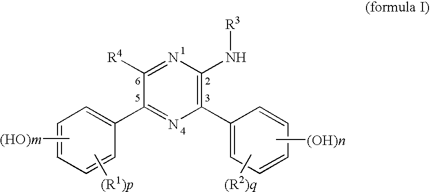
wherein:
n and m are independently 0, 1, 2, 3, 4 or 5;
p and q are independently 0, 1, 2, 3, 4 or 5;
R¹ and R² are independently H, C₁I₆ alkyl, C₁₂₋₁₈ alkyl, C₁₂₋₁₈ alkenyl, C₁₆ oxyalkyl, C₁₂₋₁₈ oxyalkyl or C₁₂₋₁₈ oxyalkenyl, fluoro, cyano, ketone, aldehyde, sulfone, nitro or any electron withdrawing group;
R³ is H or the radical of an alkylating reagent; preferably benzyl and substituted benzyl, C₁₋₆ alkyl and branched and optionally substituted C₁₋₆ alkyl with carboxyl functions and derived functions, C₁₂₋₁₈ alkyl, C₁₂₋₁₈ alkenyl;
R⁴ is H, NH₂ or NHR³;
or 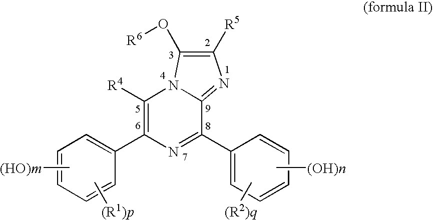
wherein:
n, m, p, q; R¹, R², R⁴ have the same definition as in formula I;
R⁵ is H or the radical of a keto-aldehyde reagent; preferably H, C₁₋₆ alkyl and branched and optionally substituted C₁₋₆ alkyl, C₁₂₋₁₈ alkyl, C₁₂₋₁₈ alkenyl, aryl and substituted aryl, benzyl and substituted benzyl;
R⁶ is H, SO₃-M+, COMe or glucoronic conjugate; or 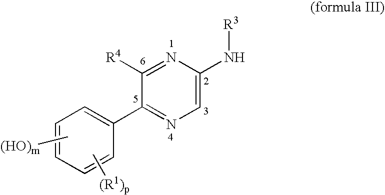
wherein:
m, p, R¹, R³, R⁴ have the same definition as in formula I
or 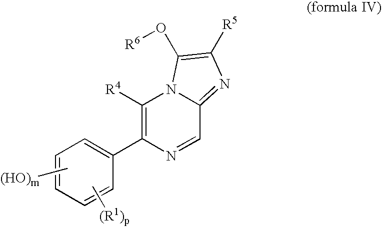
wherein:
m, p, R¹, R⁴, R⁵ have the same definition as in formula I;
R⁶ is H, SO₃-M+or COMe;
a prodrug, a pharmaceutically acceptable addition salt, a stereochemically or a tautomerically isomeric form thereof.

2. A compound as claimed in claim 1 of the general formula 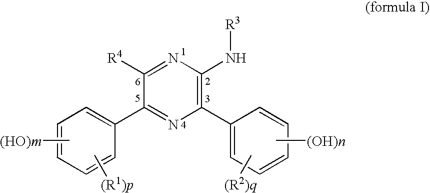
wherein:
n and m are independently 0, 1, 2, 3, 4 or 5;
p and q are independently 0, 1, 2, 3, 4 or 5;
R¹ and R² are independently H, C₁₋₆ alkyl, C₁₂₋₁₈ alkyl, C₁₂₋₁₈ alkenyl, C₁₋₆ oxyalkyl, C₁₂₋₁₈ oxyalkyl or C₁₂₋₁₈ oxyalkenyl, fluoro, cyano, ketone, aldehyde, sulfone, nitro or any electron withdrawing group;
R³ is H or the radical of an alkylating reagent; preferably benzyl and substituted benzyl, C₁₋₆ alkyl and branched and optionally substituted C₁₋₆ alkyl with carboxyl functions and derived functions, C₁₂₋₁₈ alkyl, C₁₂₋₁₈ alkenyl;
R⁴ is H, NH₂ or NHR³;

3. A compound as claimed in claim 1 of the general formula 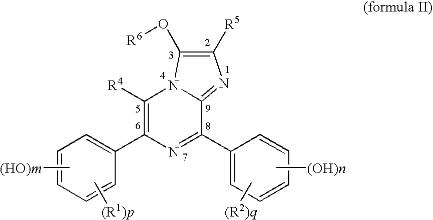
wherein:
n, m, p, q; R¹, R², R⁴ have the same definition as in formula I;
R⁵ is H or the radical of a keto-aldehyde reagent; preferably, H, C₁₋₆ alkyl and branched and optionally substituted C₁₋₆ alkyl, C₁₂₋₁₈ alkyl, C₁₂₋₁₈ alkenyl, aryl and substituted aryl, benzyl and substituted benzyl;
R⁶ is H, SO₃⁻M⁺, COMe or glucoronic conjugate.

4. A compound as claimed in claim 1 of the general formula 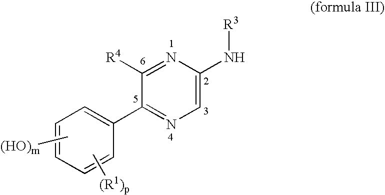
wherein:
m, p, R¹, R³, R⁴ have the same definition as in formula I.

5. A compound as claimed in claim 1 of the general formula 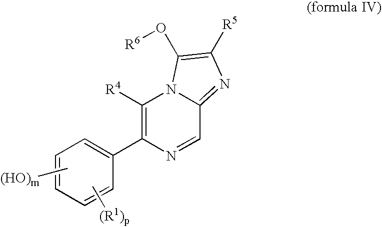
wherein:
m, p, R¹, R⁴, R⁵ have the same definition as in formula II;
R⁶ is H, SO₃-M+or COMe.

6. A compound as claimed in any of the claims 1-5, wherein n=1, preferably in the para position.

7. A compound as claimed in any of the claims 1-6, wherein m=1, preferably in the para position.

8. A compound as claimed in any of the claims 1-7, wherein m=2.

9. A compound as claimed in any of the claims 1-8, wherein n=2.

10. A compound as claimed in any of the claims 1-9, wherein R₃=H.

11. A compound as claimed in any of the claims 1-10, wherein R₄=NH₂.

12. A compound as claimed in any of the claims 1-11 having the formula
2-amino-3,5-bis(p-hydroxyphenyl)-1,4-pyrazine (CD31)
2,6-diamino-3,5-bis(p-methoxyphenyl-1,4-pyrazine (JFC26)
2,6-diamino-3,5-bis(p-hydroxyphenyl)-1,4-pyrazine (JFC28)
2-amino-3-phenyl-5-(p-methoxyphenyl)-1,4-pyrazine (CD48)
2-amino-3-phenyl-5-(p-hydroxyphenyl)-1,4-pyrazine (CD51)
2-amino-3-(p-methoxyphenyl)-5-phenyl-1,4-pyrazine (CD45)
2-amino-3-(p-hydroxyphenyl)-5-phenyl-1,4-pyrazine (CD46)
2,6-bis(1′-ethoxycarbonyl-ethylamino)-3,5-bis(p-hydroxyphenyl)-1,4-pyrazine (JFC38)
2-methyl-6,8-bis(p-hydroxyphenyl)-3,7-dihydroimidazolo[1,2-a]pyrazin-3-one (CD43)
2-methyl-6-(p-hydroxyphenyl)-8-phenyl-3,7-dihydroimidazolo[1,2-a]pyrazin-3-one (CD53)
2-methyl-6-phenyl-8-(p-hydroxyphenyl)-3,7-dihydroimidazolo[1,2-a]pyrazin-3-one (CD52)
2-Amino-5-(3,4-dimethoxyphenyl)-1,4-pyrazine (JFC48)
2-Amino-5-(3,4-dihydroxyphenyl)-1,4-pyrazine (JFC58)
2-(N-benzylamino)-5-(p-methoxyphenyl)-1,4-pyrazine (JFC55)
2-(N-benzylamino)-5-(p-hydroxyphenyl)-1,4-pyrazine (JFC71)
2-(N-benzylamino)-3,5-bis-(p-methoxyphenyl)-1,4-pyrazine (JFC72)
2-(N-benzylamino)-3,5-bis(p-hydroxyphenyl)-1,4-pyrazine (JFC73)
2-methyl-8-(3,4-dihydroxyphenyl)-3,7-dihydroimidazolo[1,2-4]pyrazin-3-one (JFC66).
2-amino-3-(3,4-dihydroxyphenyl)-5-(4-hydroxyphenyl)-1,4-pyrazine (JFC54)
2-methyl-6-(3,4-dihydroxyphenyl)-8-(4-hydroxyphenyl)-3,7-dihydroimidazolo[1,2-a]pyrazin-3-one (JFC81).

13. A compound as claimed in any of the claims 1-12 including the disclaimed compound for use of a medicament.

14. Use of a compound as claimed in any of the claims 1-13 including 2-amino-3,5-bis(p-methoxyphenyl)-1,4-pyrazine (CD29), 2-amino-5-phenyl-1,4-pyrazine (CD12), 2-amino-5-(4-methoxyphenyl)-1,4-pyrazine (CD17), 2-amino-5-(4-hydroxyphenyl)-1,4-pyrazine (CD22), 5-phenyl-2-methylamino-1,4-pyrazine and 2-amino-3,5-bis-phenyl-1,4-pyrazine and their corresponding imidazolopyrazinone compounds for the manufacture of a medicament for the prevention and/or the treatment of diseases linked to oxidative damages.

15. A pharmaceutical composition comprising a pharmaceutically acceptable carrier and an active ingredient, a therapeutically effective amount of a compound as claimed in any of the claims 1-12 including 2-amino3,5-bis(p-methoxyphenyl)-1,4-pyrazine (CD29), 2-amino-5-phenyl-1,4-pyrazine (CD12), 2-amino-5-(4-methoxyphenyl)-1,4-pyrazine (CD17), 2-amino-5-(4-hydroxyphenyl)-1,4-pyrazine (CD22), 5-phenyl-2-methylamino-1,4-pyrazine and 2-amino-3,5-bis-phenyl-1,4-pyrazine and their corresponding imidazolopyrazinone compounds.

16. Use of a compound as claimed in any of the claims 1-12 including 2-amino3,5-bis(p-methoxyphenyl)-1,4-pyrazine (CD29), 2-amino-5-phenyl-1,4-pyrazine (CD12), 2-amino-5-(4-methoxyphenyl)-1,4-pyrazine (CD17), 2-amino-5-(4-hydroxyphenyl)-1,4-pyrazine (CD22), 5-phenyl-2-methylamino-1,4-pyrazine and 2-amino-3,5-bis-phenyl-1,4-pyrazine and their corresponding imidazolopyrazinone compounds, as anti-oxidant.

17. Use of a compound as claimed in claim 16 in a diagnostic procedure.

18. Use of a compound as claimed in claim 16 in food preparation as an additive.

19. Use of a compound as claimed in claim 16 as an additive in polymers.

20. Use of a compound as claimed in claim 16 in cosmetics.

21. Method for the preparation of pyrazine compounds as claimed in any of the claims 1-12, wherein the symmetrically substituted derivatives, i.e. same aryl substituents in positions C-3 and C-5, are obtained by coupling 2-amino-3,4-dibromo-1,4-pyrazine with appropriate functionalized arylboronic acid derivatives, by using a double Suzuki-type reaction.

22. Method for the preparation of pyrazine compounds having unsymmetrically substituted derivatives, i.e. different aryl substituents in positions C-3 and C-5, by the following sequence (a) by coupling 2-amino-5-bromo-1,4-pyrazine with a first arylboronic acid derivative; (b) brominating the resulting 2-amino-5-aryl(1)-1,4-pyrazine; (c) coupling the resulting 2-amino-3-bromo-5-aryl(1)-1,4-pyrazine with a second arylboronic acid derivative resulting in the 2-amino-3-aryl(2)-5-aryl(1)-1,4-pyrazine.

23. Method for the preparation of the imidazolopyrazinones compounds wherein the method for the preparation of 2-amino-3,5-diaryl-1,4-pyrazines as claimed in claims 21 and 22 is continued by condensation with keto-aldehyde reagents under acidic conditions.

24. Method for the preparation of the pyrazine compounds as claimed in any of the claims 1-12, by (a) coupling 2-amino-5-bromo-1,4-pyrazine with a first arylboronic acid derivative; if necessary when two aryl substituents are desired bromating the resulting 2-amino-5-aryl-1,4-pyrazine and coupling the resulting 2-amino-3-bromo-5-aryl-1,4-pyrazine with a second arylboronic acid derivative resulting in the 2-amino-3-aryl-5-aryl-1,4-pyrazine.

25. Method for the preparation of the pyrazine compounds as claimed in claim 24, continued by condensation with keto-aldehyde reagents under acidic conditions.

26. Method for the preparation of the pyrazine compounds as claimed in claims 21 and 22, continued by the N-alkylation of the 2-amino (or 2,4-diamino) function(s).

27. A compound of the general formula, 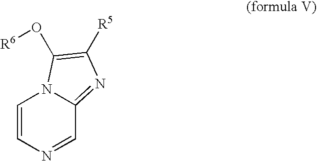
wherein R⁵ and R⁶ are defined as for formula II;
a pharmaceutically acceptable addition salt, a stereochemically or a tautomerically isomeric form thereof for use as a medicament in particular for use as an anti-oxidant.

28. A compound according to claim 27, wherein R₆ is H and R₅ is Me, Ph, tBu, Et, PhpCl, PhpOMe, CH₂Ph, CH₂PhpOMe, CH₂PhpCF₃.

29. Anti-oxidant compound generating upon oxidation a second anti-oxidant compound and a third compound.

30. Anti-oxidant compound according to claim 29, having the general formula II of claim 3 or formula IV of claim 5.

31. Anti-oxidant compound according to claim 29 or 30, wherein the second anti-oxidant compound is having the general formula I of claim 2 and formula ill of claim 4.

32. Anti-oxidant compound according to any of the claims 29-31, wherein the third compound is an anti-oxidant or an anti-inflammatory agent.
